(19) United States
(12) Patent Application Publication
(10) Pub. No.: US 2019/0144855 A1
(43) Pub. Date: May 16, 2019

(54) FOCUSED ACOUSTICS MEDIATED NUCLEIC ACID LIGATION

(71) Applicant: Covaris, Inc., Woburn, MA (US)

(72) Inventors: Lauren E. Jansen, East Boston, MA (US); Hans-Ulrich Thomann, Stow, MA (US); Eugenio Daviso, Pelham, NH (US); James A. Laugharn, JR., Boston, MA (US)

(73) Assignee: Covaris, Inc., Woburn, MA (US)

(21) Appl. No.: 16/182,740

(22) Filed: Nov. 7, 2018

Related U.S. Application Data
(60) Provisional application No. 62/583,099, filed on Nov. 8, 2017.

(51) Int. Cl. C12N 15/10; C12P 19/34

(52) U.S. Cl. CPC C12N 15/1086; C12P 19/34

(57) ABSTRACT

System and method for enhancing ligation of nucleic acid portions using focused acoustic energy.

### CROSS REFERENCE TO RELATED APPLICATION

This application claims the benefit of U.S. Provisional Application No. 62/583,099, filed Nov. 8, 2017, which is hereby incorporated by reference in its entirety.

### BACKGROUND

### 1. Field of Invention

Systems and methods of acoustic processing are generally disclosed.

### 2. Related Art

Ultrasonics have been utilized for a variety of diagnostic, therapeutic, and research purposes. Some uses of sonic or acoustic energy in materials processing include “sonication,” an unrefined process of mechanical disruption involving the direct immersion of an acoustic source emitting unfocused energy in the kilohertz (“kHz”) range into a fluid suspension of the material being treated. Such sonic energy often does not reach a target in an effective dose because the energy is scattered, absorbed, and/or not properly aligned with the target. Sonication has also hit limits on effectiveness when applied to higher sample volumes or continuous process streams. There are also specific clinical examples of the utilization of therapeutic ultrasound (e.g., lithotripsy) and of diagnostic ultrasound (e.g., fetal imaging). However, ultrasonics have generally not been controlled in a manner so as to provide automated, broad range, precise materials processing or reaction control mechanisms.

### SUMMARY

Tools and methods are disclosed that aid in enhancing enzymatic reactions such as DNA and other nucleic acid ligation reactions and other enzymatic reactions that require stable ternary complexes.

Enzymatic reactions are used to amplify and manipulate natural and/or synthetic DNA constructs for many molecular biology applications. Two major fields that exploit enzymatic reactions are next generation sequencing (NGS) and synthetic biology. In prepping DNA for NGS, enzymes are used to ligate adaptors to DNA and amplify the DNA. In synthetic biology, enzymes are used to make precise cuts in DNA, to remove or add nucleotides, and to piece together constructs. Though many processes are made possible and are improved via enzymatic reactions, they can also be limited by lack of efficiency. Low yield can be overcome with long incubation times and the use of excess reagents, but this can add expense to the reaction and requires additional downstream clean up. Aspects of the invention overcome these challenges, e.g., increasing the product yield of enzymatic reactions, by creating a specialized reaction environment using focused acoustic energy

In some embodiments, aspects of the invention focus on improving specific steps in an NGS workflow. NGS library prep starts with isolated DNA and ends with a NGS-ready, adapter-ligated, and possibly size-selected fragment library. A pre-requisite for the majority of NGS applications is DNA fragmentation, and focused acoustic energy can be used for this process, e.g., as taught in US20160102329. After DNA fragmentation (also referred to as DNA shearing), fragments may undergo size-selection to exclude short, undesirable fragments. This is achieved by binding the sheared DNA population to magnetic beads, washing away undesired fragment lengths, and subsequently eluting the desired fragment population off the beads. Single-stranded fragment overhangs formed during the acoustic shearing are then blunt end repaired and 5′ phosphorylated, followed by an addition of a 3′ terminal A by so-called non-templated A addition with the Taq DNA polymerase. These preparation steps involve enzymatic reactions in which a single substrate is modified, i.e., removal or addition of nucleotides, and 5′ phosphorylation.

The next step in the NGS library construction process is adapter ligation. This step is by far the most complex in the NGS workflow, making it a challenge in the generation of enough adapter ligated fragments needed to perform NGS. During this step, two adapters must be joined to both ends of a DNA fragment. Fragments and adapters have only a single nucleotide overhang (3′ A on the fragment and 3′ T on the linker, or adapter), thus decreasing the probability that the two molecules will ‘stick’ together. The ligation is therefore an inefficient process that requires special conditions to synthesize sufficient quantities of double-ligated fragments.

Adapter ligation for NGS library preparations are typically performed at room temperature and incubation times last usually 20-30 minutes (e.g., as used with the Accel-NGS 2S Plus kit by SWIFT Biosciences, Ann Arbor, Mich.; NxSeq UltraLow DNA Library Kit by Lucigen Corporation, Middleton, Wis.; or TruSEQ DNA PCR—free kit by Illumina, San Diego, Calif.). High yield of 2-adaptor fragments for NGS is key to reducing the need to amplify DNA via PCR, which introduces the most error into sequencing reads. Ligation is a major bottleneck in the library prep pipeline because of both low yield of 2-apaptor fragments and the need for multiple clean-ups of non-ligated adaptors due to the use of a high excess of adaptors in the reaction. (Because of the poor binding of adaptors to DNA fragments, a very high concentration of adaptors, such as 50 times or more than the DNA fragments on a molar basis, are used to help achieve the desired 2-adaptor fragment yield.) Either increasing the yield of 2-adapter fragments from the ligation reaction and/or decreasing the need for excess adaptors in the reaction would ultimately lead to less error in downstream sequencing.

Thus, improvements to ligation efficiency will not only increase the yields of linker-ligated products (especially those that are linker ligated at both ends of the fragment), but will also reduce the number of linkers needed to drive the reaction forward. Currently, to push DNA ligation reactions towards double ligated products, reactions contain close to 50-100-fold molar excess of linkers to fragment. This linker excess has implications for downstream processing because these linkers need to be removed before ligation products can be quantitated and used for populating flow cells (e.g., using the Illumina NGS platform) or beads (e.g., using the IonTorrent or 454 NGS platforms). Linker removal is done by size-selective binding to magnetic beads. However, complete linker removal is almost impossible, especially when the size difference of linker-ligated fragments to linkers is relatively small. Incomplete linker removal can result in linker carryover during sequencing (NGS based analysis involves pooling of multiple linker-ligated fragment pools, each with its own sequence tag) and misidentification of samples. This problem is known as Index Switching or Index Hopping. A simple solution to linker carry-over is to reduce the linker to fragment ratio in the ligation reaction. Currently, this would reduce the formation of double ligated fragments significantly, simply due to unfavorable reaction kinetics: by lowering linker concentrations, possible collisions between fragment and linker ends are also reduced.

DNA ligation provides a perfect model system for ternary enzyme substrate complexes. In such complexes, two substrates and the enzyme must be present to form a product. DNA ligases were discovered in 1967 and these enzymes are important tools in molecular biology, enabling essential in vitro assays like cloning and NGS analysis. Two major families of DNA ligases are known, i.e., ATP-dependent and NAD-dependent ligases, which are found in eukaryotes and prokaryotes, respectively. However, the mechanism of DNA ligases is a three-step process, regardless of the different co-factors. Ligation starts with activation of the enzyme by the cofactor (ATP or NAD), followed by activation of the 5′ end of a DNA strand and subsequent joining of the activated 5′ end with an adjacent 3′ end of another DNA strand.

In vivo DNA ligases are designed to join single-stranded nicks in double-stranded DNA. The two ends are already fixed in place, unlike in an in vitro ligation reaction where two independent fragment ends must be joined. In contrast to nick-ligation/nick repair, ligating two isolated fragments of DNA molecules is much less efficient because two substrates, i.e., each DNA fragment end to be joined, must find each other first. Thus, increasing substrate concentrations either of both or one substrate is a common choice to overcome this inefficiency in vitro. Incubation is another factor that limits in vitro ligation efficiency. The optimal enzyme activity of commercially available and commonly used T4, T3 or T7 DNA ligases is 25° C. However, at this temperature molecular movement is high, reducing the chances that two substrates will ‘find’ each other. Therefore, ligation reactions are commonly performed at lower temperatures such as 4 or 16 degrees C. to reduce temperature-based molecular movement. Naturally enzyme activity is significantly reduced at this temperature, so prolonging incubation times to overnight is a common practice for in vitro ligation.

Another factor impacting ligation reactions is the nature of the fragment ends. Sticky ends are a much better substrate for DNA ligases as compared to blunt ends or compatible single nucleotide overhangs (e.g., T/A cloning). Sticky ends are 3′ or 5′ single stranded, compatible overhangs on DNA fragments such as those commonly created by restriction enzyme digests of DNA, enabling them to hybridize and stabilize the complex of the two to be joined DNA fragments. On the other hand, blunt ends or single nucleotide overhangs rely on end-to-end collisions.

Transient stability of such DNA-DNA end to end fragment complexes is a major contributor to DNA ligation efficiency. The addition of viscosity increasing agents and/or so-called nucleic acid crowding agents, such as poly(ethylene glycol-6000) (PEG-6000), glycerol, glycogen, albumin, Ficoll has been shown to enhance DNA ligation reactions. Macromolecular crowding increases local concentrations of DNA in ligation reactions, thereby increasing the probability of two DNA fragments being in close vicinity. Commercially DNA ligase kits, as well as all NGS library preparation kits (Table 1) may contain such agents, mainly PEG-6000 due to its compatibility with downstream processing of ligation products, in their ligase reaction buffer. This reduces the reaction time significantly (30 to 60 min versus overnight) and allows the reaction temperature to be more favorable for the enzyme (20° C. versus 12° C.). However, macromolecular crowding also greatly reduces the diffusion and molecular movement of DNA substrates as well as enzymes, thereby impacting the possible turnover rate by local depletion of substrate. Table 1 lists the companies that offer commercial ligation kits and notes if those companies employ molecular crowding in the reaction buffer.

TABLE 1
List of major suppliers of Ligation and NGS Library prep kits.

| Manufacturer | Kit | Comment |
|---|---|---|
| New England | Quick and standard ligation | Quick Ligation and NGS |
| Biolabs (NEB) | kits; ligases; NGS library | Library Ligation buffers |
| | prtep kits | contain PEG |
| Sigma-Aldrich | Standard and Rapid Ligation | Rapid Ligation and NGS |
| | kits; NGS library prep kits | Library Ligation buffers |
| | | contain PEG |
| Promega | Standard and Rapid | Rapid Ligation buffers |
| | | contain PEG |
| ThermoFisher | Rapid Ligation and NGS | Buffers contain PEG |
| | Library kits | |
| Qiagen | NGS Library Prep kits | |
| Illumina | NGS Library Prep kits | |
| KAPA (Roche) | NGS Library Prep kits | |
| Zymo Research | NGS Library Prep kits | |
| Lucigen | NGS Library Prep kits, | |
| | circularization Ligase, Quick | |
| | ligase | |
| SIFT | NGS Library Prep kits | |
| Biosciences | | |

While being key to binding adapters to DNA and other nucleic acid fragments, ligation is an essential step for other applications such as DNA circularization, which can be used in gene cloning and targeted sequencing in library prep for NGS. In such processes, nucleic acid fragments may be ligated together (self-ligation) in addition to ligating adaptors or linkers to nucleic acid fragments. One example of targeted NGS is CircleSeq, an in vitro screen of off-target binding of CRISPR guide RNA's. The CircleSeq workflow involves ligation of adapters to fragmented genomic DNA followed by circularization (self-ligation). Circularized DNA is then introduced to a guide RNA and the Cas-9 enzyme and fragments harboring off- and on-target loci are cleaved. These cleaved sequences are then available for a third ligation of adapters for NGS, where the remaining circular DNA is removed from the reaction. While this could be a powerful tool in the clinical setting, the use of multiple ligation reactions, and particularly the process of circularization, makes this technique extremely inefficient.

The inventors have found that focused acoustic energy enhances enzymatic efficiencies for processes that require formation of ternary or quaternary complexes, including those that are carried out in the presence of crowding agents. That is, focused acoustic energy can enhance not only adapter ligation reactions, but other ligation reactions (DNA circularization and/or RNA/RNA during splicing for example), as well as acetylation of proteins (such as by serine acetyl CoA transferases), and loading of tRNA with aminoacids (by aminoacid-tRNA synthetases). In some embodiments, the biggest increase in efficiency may be achievable for reactions that do not employ a vast excess of adaptor substrates in the reaction and where the rate of the reaction is typically dictated by diffusion. An example is the splint ligation, which is used to synthesize long RNA molecules. The inventors have coined the term “micromixing” to refer to the effect of suitably configured focused acoustic energy that overcomes limitations to typical ligation reactions by ‘refreshing’ the local concentrations of adaptor or other substrate to the ligation target, thereby avoiding ‘stalling’ of the ligation reaction common in prior processes.

Another application where focused acoustic energy can be useful is the assembly of multiple pieces of DNA, which can be done using a series of ligation reactions. This application enables the creation of larger, more complex constructs of DNA used to direct cell function. Recent efforts to recreate fully synthetic genomes, or find a minimal genome, are examples where many pieces of DNA need to be assembled in a series of reactions. A Gibson Assembly is a one-batch reaction that assembles multiple pieces of DNA and is used to create these genome-sized structures: A T5 exonuclease, a DNA polymerase, and a DNA ligase are incubated with overlapping DNA molecules to assemble DNA fragments together. This reaction employs several enzymes that rely on binding, modifying and joining/ligating more than one substrate in the same enzyme-substrate complex. Currently, Gibson's can efficiently assemble 2-4 pieces of DNA at one time. While this eliminates the need for specific restriction enzymes and is a lot faster than previous cloning techniques, assembling a genome requires multiple Gibson assemblies. Improving ligation efficiency in the reaction is one way to increase the number of fragments that can be assembled in a single reaction, but there are also many enzymes in this reaction that benefit from focused acoustic energy. Additionally, the ability to increase the number of fragments at the same time saves time and money for research.

Other advantages and novel features of the invention will become apparent from the following detailed description of various non-limiting embodiments when considered in conjunction with the accompanying figures and claims.

### BRIEF DESCRIPTION OF THE DRAWINGS

Aspects of the invention are described with reference to the following drawings in which numerals reference like elements, and wherein:

FIG. 1 shows steps in a process for preparing DNA for NGS sequencing;

FIG. 2 shows a perspective view of an acoustic treatment apparatus that incorporates one or more aspects of the invention;

FIG. 3 illustrates DNA fragment and adapter distribution in an experimental model;

FIG. 4 shows DNA fragment and adapter distribution of the FIG. 3 embodiment after introduction of a ligating enzyme;

FIG. 5 shows 1-linker and 2-linker product percentage yield in experiments employing different ligation reaction times, temperatures, crowding agent and/or focused acoustic energy;

FIG. 6 shows 1-linker and 2-linker product percentage yield in experiments employing different levels of focused acoustic energy;

FIG. 7 shows 2-linker product percentage yield in experiments employing different levels of crowding agent by weight percentage and different types of crowding agents;

FIG. 8 shows 1-linker and 2-linker product percentage yield in experiments employing different levels of crowding agent, enzyme and focused acoustic energy;

FIG. 9 shows 2-linker product yield in a conventional ligating protocol both with and without the use of focused acoustic energy; and

FIG. 10 shows 1-linker and 2-linker product yield in experiments employing different levels of initial adapter fragment.

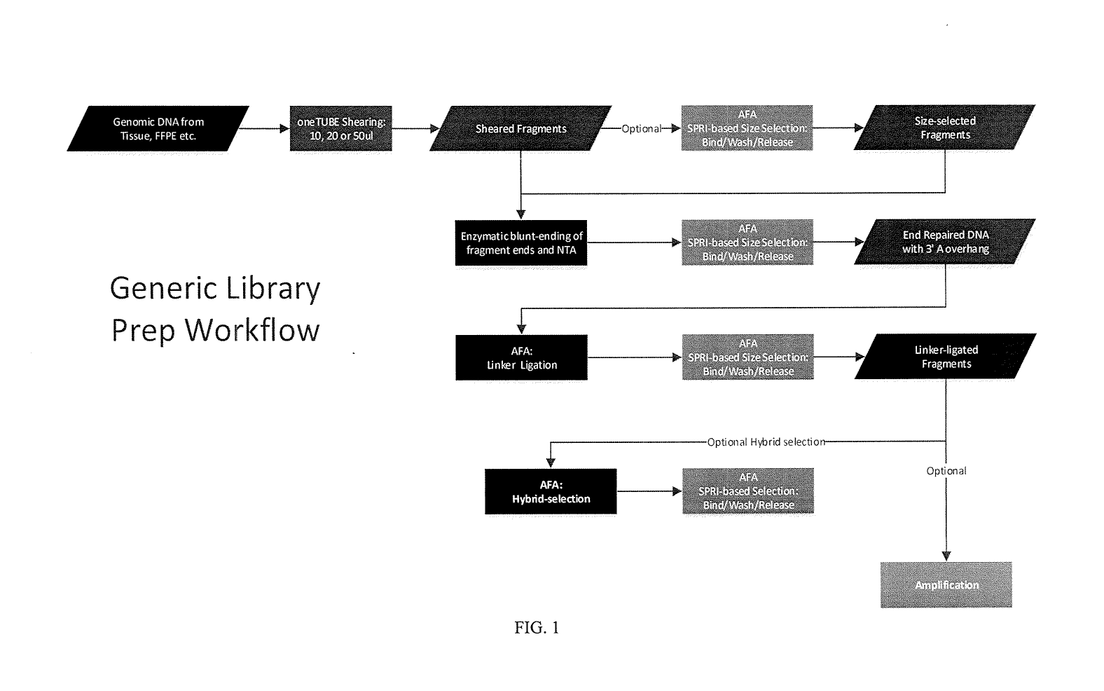

### DETAILED DESCRIPTION

“Sonic energy” or “acoustic energy” as used herein is intended to encompass such terms as acoustic waves, acoustic pulses, ultrasonic energy, ultrasonic waves, ultrasound, shock waves, sound energy, sound waves, sonic pulses, pulses, waves, or any other grammatical form of these terms, as well as any other type of energy that has similar characteristics to sonic energy.

Acoustic energy, including focused acoustic energy, can be used for treating a wide variety of different types of sample material, and can cause various different effects on the sample material. Often, the physical characteristics of the acoustic energy must be carefully selected and controlled to achieve a desired treatment effect. For example, acoustic energy of a desired wavelength, peak incident power (PIP), duty cycle, cycles per burst and/or other characteristics may be emitted from an acoustic energy source and used to treat a material at a treatment location, e.g., to enhance enzyme-assisted ligation reactions. However, treatment with acoustic energy can cause heating of the sample, and in some cases, acoustic treatment of a sample material is ideally done at or below a specified temperature. As just one example, exposing a sample material to increased temperatures may cause damage to the sample material, such as denaturing of proteins or other degradation of compounds. Sample heating can be reduced by reducing the overall power of the acoustic energy used, but reduction in acoustic energy power may in certain circumstances significantly increase treatment time at best, and be completely ineffective in treating a sample at worst. Thus, preventing exposure of some sample materials to temperatures over a desired threshold may be desirable or necessary, especially when employing relatively high acoustic energy power or total energy levels.

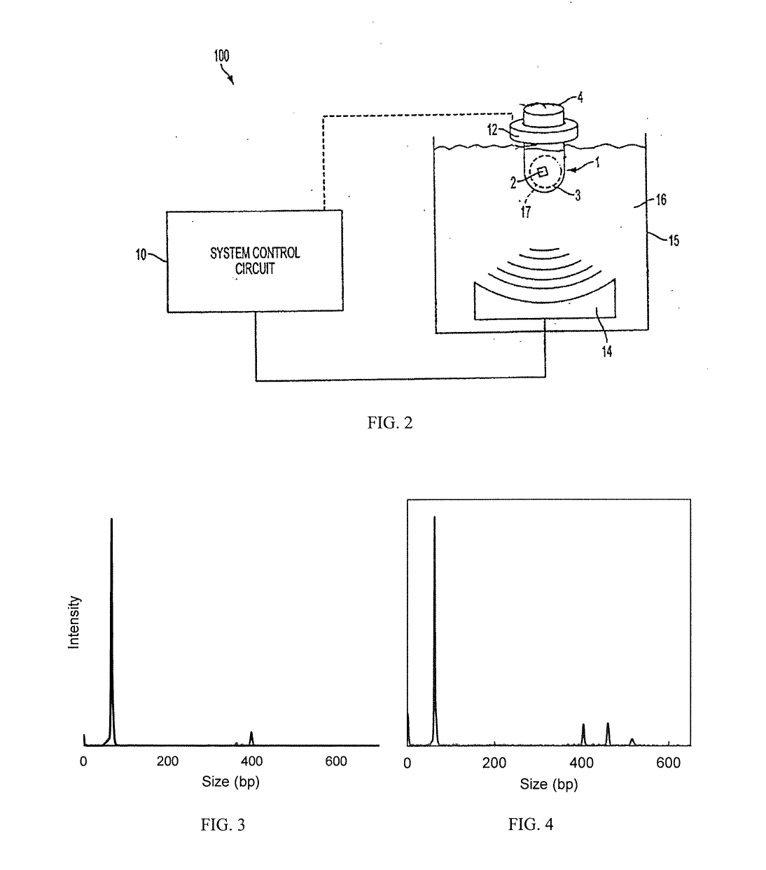

FIG. 2 shows a schematic block diagram of an acoustic treatment system 100 that may be used to provide focused acoustic treatment in one or more embodiments, e.g., to enhance nucleic acid ligation reactions. It should be understood that although embodiments described herein may include most or all aspects of the invention, aspects of the invention may be used alone or in any suitable combination with other aspects of the invention. In this illustrative embodiment, the acoustic treatment system 100 includes an acoustic energy source with an acoustic transducer 14 (e.g., including one or more piezoelectric elements) that is capable of generating an acoustic field (e.g., at a focal zone 17) suitable to cause mixing, e.g., caused by cavitation, and/or other effects in a sample 1 contained in a vessel 4. The sample 1 may include “solid” particles, such as cells, or other material 2, such as DNA or other nucleic acid material, one or more enzymes, etc. and/or liquid 3, such as liquid reagents, water, a crowding agent, etc. The vessel 4 may have any suitable size or other arrangement, e.g., may be a glass or metal tube, a plastic container, a well in a microtiter plate, a vial, or other, and may be supported at a location by a vessel holder 12. Although a vessel holder 12 is not necessarily required, the vessel holder 12 may interface with a control circuit 10 so that the vessel 4 and the sample in the vessel 4 is positioned in a known location relative to an acoustic field, for example, at least partially within a focal zone 17 of acoustic energy. The vessel holder 12 may be arranged to support the vessel 4 in a single location, or may be arranged to move the vessel 4, e.g., using a robotic system, movable stage or other drive system. In this embodiment, the vessel 4 is a polymer tube having an internal volume of 50 microliters or less (e.g., 20 microliters), but it should be understood that the vessel 4 may have other suitable shapes, sizes, materials, or other features, as discussed more below. For example, the vessel 4 may be a cylindrical tube with a flat bottom and a threaded top end to receive a cap, may include a cylindrical collar with a depending flexible bag-like portion to hold a sample, may be a single well in a multiwell plate, may be a cube-shaped vessel, or may be of any other suitable arrangement. The vessel 4 may be formed of glass, plastic, metal, composites, and/or any suitable combinations of materials, and formed by any suitable process, such as molding, machining, stamping, and/or a combination of processes.

As can be seen in FIG. 2, a container 15 may contain the acoustic transducer 14 or other acoustic energy source, the vessel 4 as well as a coupling medium 16. The container 15 may take any suitable size, shape or other configuration, and may be made of any suitable material or combination of materials (such as metal, plastic, composites, etc.). In this illustrative embodiment, the container 15 has a jar- or can-like configuration with an opening arranged to permit access to an internal volume of the container 15. The container 15 may be arranged to hold any suitable coupling medium 16, such as water or another liquid, gas (e.g., air, inert gas), gel (e.g., silicone), solid (e.g., elastomeric material), semi-solid, and/or a combination of such components, which transmits acoustic energy from the transducer 14 to the treatment chamber 4. The acoustic energy source 14 and the coupling medium 16 (such as water or other liquid, or optionally a solid material) may be positioned in the container 15, e.g., with the acoustic energy source 14 near a bottom of the container 15. (If the coupling material 16 is solid, the container 15 and the coupling medium 16 may be essentially integrated with each other, with the coupling medium 16 essentially functioning as an acoustic coupling as well as a physical attachment of the acoustic source 14 and the vessel 4.) The vessel 4 can be lowered into the container 15, e.g., so that the vessel 4 is partially or completely submerged in the coupling medium 16. The coupling medium 16 may function as both an acoustic coupling medium, e.g., to transmit acoustic energy from the acoustic energy source 14 to the vessel 4, as well as a thermal coupling medium, e.g., to accept heat energy from the vessel 4. In other embodiments, the thermal and acoustic coupling medium may be separate, e.g., where the vessel 4 is provided with a cooling jacket.

Under the control of the control circuit 10 (described in more detail below), the acoustic transducer 14 may produce acoustic energy within a frequency range of between about 100 kilohertz and about 100 megahertz such that the focal zone 17 has a width of about 2 centimeters or less. The focal zone 17 of the acoustic energy may be any suitable shape, such as spherical, ellipsoidal, rod-shaped, or column-shaped, for example, and be positioned at the sample 1. The focal zone 17 may be larger than the sample volume, or may be smaller than the sample volume, as shown in FIG. 2. U.S. Pat. Nos. 6,948,843 and 6,719,449 are incorporated by reference herein for details regarding the construction and operation of an acoustic transducer and its control. The focal zone may be stationary relative to the sample, or it may move relative to the sample.

In some embodiments, the transducer can be formed of a piezoelectric material, such as a piezoelectric ceramic. The ceramic may be fabricated as a “dome”, which tends to focus the energy. One application of such materials is in sound reproduction; however, as used herein, the frequency is generally much higher and the piezoelectric material would be typically overdriven, that is driven by a voltage beyond the linear region of mechanical response to voltage change, to sharpen the pulses. Typically, these domes have a longer focal length than that found in lithotriptic systems, for example, about 20 cm versus about 10 cm focal length. Ceramic domes can be damped to prevent ringing or undamped to increase power output. The response may be linear if not overdriven. The high-energy focus zone 17 of one of these domes is typically cigar-shaped. At 1 MHz, the focal zone 17 is about 6 cm long and about 2 cm wide for a 20 cm dome, or about 15 mm long and about 3 mm wide for a 10 cm dome. The peak positive pressure obtained from such systems at the focal zone 17 is about 1 MPa (mega Pascal) to about 10 MPa pressure, or about 150 PSI (pounds per square inch) to about 1500 PSI, depending on the driving voltage. The focal zone 17, defined as having an acoustic intensity within about 6 dB of the peak acoustic intensity, is formed around the geometric focal point. It is also possible to generate a line-shaped focal zone, e.g., that spans the width of a multi-well plate and enables the system 1 to treat multiple wells simultaneously.

To control an acoustic transducer 14, the system control circuit 10 may provide control signals to a load current control circuit, which controls a load current in a winding of a transformer. Based on the load current, the transformer may output a drive signal to a matching network, which is coupled to the acoustic transducer 14 and provides suitable signals for the transducer 14 to produce desired acoustic energy. Moreover, the system control circuit 10 may control various other acoustic treatment system 100 functions, such as positioning of the vessel 4 and/or acoustic transducer 14 (e.g., by controlling the vessel holder 12 to suitably move and hold the vessel 4 in a desired location), receiving operator input (such as commands for system operation by employing a user interface), outputting information (e.g., to a visible display screen, indicator lights, sample treatment status information in electronic data form, and so on), and others. Thus, the system control circuit 10 may include any suitable components to perform desired control, communication and/or other functions. For example, the system control circuit 10 may include one or more general purpose computers, a network of computers, one or more microprocessors, etc. for performing data processing functions, one or more memories for storing data and/or operating instructions (e.g., including volatile and/or non-volatile memories such as optical disks and disk drives, semiconductor memory, magnetic tape or disk memories, and so on), communication buses or other communication devices for wired or wireless communication (e.g., including various wires, switches, connectors, Ethernet communication devices, WLAN communication devices, and so on), software or other computer-executable instructions (e.g., including instructions for carrying out functions related to controlling the load current control circuit as described above and other components), a power supply or other power source (such as a plug for mating with an electrical outlet, batteries, transformers, etc.), relays and/or other switching devices, mechanical linkages, one or more sensors or data input devices (such as a sensor to detect a temperature and/or presence of the medium 16, a video camera or other imaging device to capture and analyze image information regarding the vessel 4 or other components, position sensors to indicate positions of the acoustic transducer 14 and/or the vessel 4, and so on), user data input devices (such as buttons, dials, knobs, a keyboard, a touch screen or other), information display devices (such as an LCD display, indicator lights, a printer, etc.), and/or other components for providing desired input/output and control functions. Also, the control circuit 10 may include one or more components to detect and control a temperature of the coupling medium 16, such as a refrigeration system to chill the coupling medium 16, a degassing system to remove dissolved gas from the coupling medium 16, etc. Circulating the coupling medium 16 may allow the control circuit 10 to remove portions of the coupling medium 16 from the container 15 for processing, such as degassing, chilling, replacement, addition of compounds, etc.

Although not necessarily critical to employing aspects of the invention, in some embodiments, sample treatment control may include a feedback loop for regulating at least one of acoustic energy location, frequency, pattern, intensity, duration, and/or absorbed dose of the acoustic energy to achieve the desired result of acoustic treatment. One or more sensors may be employed by the control circuit 10 to sense parameters of the acoustic energy emitted by the transducer 14 and/or of the sample material 1, and the control circuit 10 may adjust parameters of the acoustic energy and/or of the sample material 1 (such as flow rate, concentration, etc.) accordingly. Thus, control of the acoustic energy source may be performed by a system control unit using a feedback control mechanism so that any of accuracy, reproducibility, speed of processing, control of temperature, provision of uniformity of exposure to sonic pulses, sensing of degree of completion of processing, monitoring of cavitation, and control of beam properties (including intensity, frequency, degree of focusing, wave train pattern, and position), can enhance performance of the treatment system. A variety of sensors or sensed properties may be used by the control circuit for providing input for feedback control. These properties can include sensing of temperature of the sample material; sonic beam intensity; pressure; coupling medium properties including temperature, salinity, and polarity; sample material position; conductivity, impedance, inductance, and/or the magnetic equivalents of these properties, and optical or visual properties of the sample material. These optical properties, which may be detected by a sensor typically in the visible, IR, and UV ranges, may include apparent color, emission, absorption, fluorescence, phosphorescence, scattering, particle size, laser/Doppler fluid and particle velocities, and effective viscosity. Sample integrity and/or comminution can be sensed with a pattern analysis of an optical signal from the sensor. Particle size, solubility level, physical uniformity and the form of particles could all be measured using instrumentation either fully standalone sampling of the fluid and providing a feedback signal, or integrated directly with the focused acoustical system via measurement interface points such as an optical window. Any sensed property or combination thereof can serve as input into a control system. The feedback can be used to control any output of the system, for example beam properties, sample position or flow in the chamber, treatment duration, and losses of energy at boundaries and in transit via reflection, dispersion, diffraction, absorption, dephasing and detuning.

The desired result of acoustic treatment, which may be achieved or enhanced by use of ultrasonic wavetrains, can be, without limitation, heating the sample, cooling the sample, fluidizing the sample, micronizing the sample, mixing the sample, stirring the sample, disrupting the sample, permeabilizing a component of the sample, forming a nanoemulsion or nano formulation, enhancing a reaction in the sample, solubilizing, sterilizing the sample, lysing, extracting, comminuting, catalyzing, and/or selectively degrading at least a portion of a sample. In embodiments specifically discussed herein, specialized mixing of the sample is particularly effective in enhancing ligation reactions. Sonic waves may also enhance filtration, fluid flow in conduits, and fluidization of suspensions. Processes in accordance with the present disclosure may be synthetic, analytic, or simply facilitative of other processes such as stirring.

### Example 1: Ligation Measured Using Fragment Analysis

Several experiments, or examples, were conducted to illustrate enhancements to ligation reactions provided by the suitable use of focused acoustic energy, e.g., using a system like that shown in FIG. 2. To provide model substrates for such ligation examples, reaction fragments were synthesized via PCR. 60 bp primers were designed against the plasmid pUC57 (Forward: GCTCTTGATCCGGCAAACAA; Reverse: GTATCATTGCAGCACTGGGG for a 396 bp fragment) and ordered desalted (IDT, Coralville, Iowa) as 5′ phosphorylated oligonucleotides. 0.5 ng of the pUC57 plasmid was mixed with 200 nM of each 60 bp primer and PCR amplification performed using the Platinum PCR SuperMix High Fidelity (ThermoFisher, Waltham, Mass.). Amplification occurred over 35 cycles, with denaturation at 95° C. for 10 seconds, annealing at 56° C. for 30 seconds, and extension at 68° C. for 30 seconds (0.1° C./second) on in a Nexus GSX1 thermocycler (Eppendorf, Hamburg, Germany). Final incubation at 72° C. for 30 minutes was used to add 3′ dA overhangs (Clark, 1988). The DNA fragments were purified above 150 bp using the Select-a-Size DNA Clean and Concentrator kit (Zymo Research, Irvine, Ca), according to the manufacturer's instructions. Size and purity were validated on a 1% agarose gel run on the E-Gel Electrophoresis System (ThermoFisher).

For synthesis of a 60 bp linker fragment, two oligonucleotides were synthesized and gel-purified by IDT. L1: 5′-TCT AGC CTT CTC GCA GCA CAT CCC TTT CTC ACA TCT AGA GCC ACC AGC GGC ATA GTA AT-3′ and L2: 5′-pTT ACT ATG CCG CTG GTG GCT CTA GAT GTG AGA AAG GGA TGT GCT GCG AGA AGG CTA GA p-3′. To synthesize a double-stranded linker, equimolar quantities of L1 and L2 were annealed in a solution containing 10 mM Tris-HCl, pH 7.5, 125 mM NaCl by heating to 95° C. for 10 seconds followed by slow cooling to 60° C. for 10 seconds and 10° C. for 10 seconds (0.1° C./second) in a Nexus GSX1 thermocycler (Eppendorf). The linker contains a 3′ dT overhang (L1), and on the complementary strand a 5′ as well as a 3′ phosphate (L2). The 3′ dT overhang provides compatibility for ligation to a fragment with a 3′ A overhang. The 3′ phosphate blocks this end of the hybridized double-stranded linker to form linker dimers.

Fragment analysis was done using capillary electrophoresis performed on a 48-capillary fragment analyzer (Advanced Analytical Technologies, Ankeny, Iowa) using a High Sensitivity fragment gel with a range of 1-6000 bp (Advanced Analytical). FIG. 3 shows results of electrophoresis run on the mixture of DNA fragments and linkers above prior to the introduction of an enzyme to mediate ligation, whereas FIG. 4 shows results after the addition of a ligation enzyme. As seen in FIG. 3, when no ligase is present there are clear peaks at 60 bp and 396 bp, corresponding to the linkers and fragments, respectively. That is, little or no ligation of linkers to fragments has occurred. However, as shown in FIG. 4, when a ligase (e.g., T4 ligase) is present, two new peaks appear at about 460 bp and about 520 bp, corresponding to fragments with 1- and 2-linkers attached. As seen in FIG. 4, the ligation of 2-linkers to a DNA fragment has a relatively low yield even though a huge excess of linkers are used in the reaction. The magnitude of the peaks in FIG. 4 is relative to the product yield, so a custom Matlab code was written to calculate the yield provided from each ligation reaction (Mathworks, Natick, Mass.).

### Example 2: Ligation Improved with Focused Acoustic Energy

Unless stated otherwise, all ligations were performed with 50 ng of 396 bp fragment, 0.12 μm of 60 bp linkers, and 1 μL of T4 ligase (1 U/μL, Sigma) in a 20 μL reaction buffer containing 10 mM Tris HCL (AmericanBio, Natick, Mass.), 5 mM MgCl2 (Sigma-Aldrich, St. Louis, Mo.), 0.2 mM ATP (NEB, Ipswich, Mass.), and 1 mM DTT (Sigma). When polyethylene glycol (PEG) is added to the ligation buffer it is a 6 k PEG (Sigma) at 12.5 wt %, unless stated differently. Ligation reactions were done in a Covaris oneTUBE-10 and focused acoustic energy was performed on an E220 model machine (Covaris, Woburn, Mass.) set to give a 1 second burst of focused acoustic energy at 20 peak incident power (PIP), 50% duty factor, and 100 cycles per burst every minute.

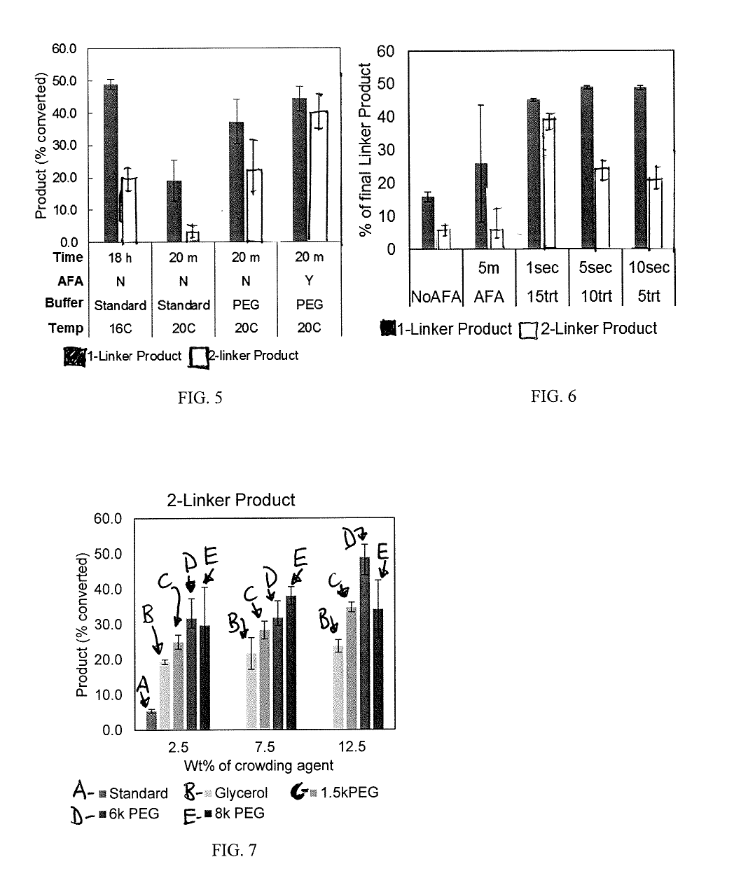

As previously discussed, manipulating temperature/time and adding a crowding agent can be used to improve the product yield for a ligation reaction. FIG. 5 shows results of four experiments employing different total reaction times (18 hours vs. 20 minutes), a crowding agent or not (use of PEG or not), different reaction temperatures (16 degrees C. vs. 20 degrees C.), and focused acoustic energy or not (AFA indicates focused acoustic energy). FIG. 5 indicates the percentage yield of 2-linker fragments that are produced for each experiment. As shown in FIG. 5, the use of PEG and increased reaction temperature are both effective ways to increase the yield of a 2-linker product compared to a standard ligation reaction in which a relatively lower temperature (16 degrees C.) and no crowding agent (no PEG) are employed. However, even the best performing experiment that does not include the use of focused acoustic energy has a maximum 2-linker product yield of about 20%, which is still limiting for certain applications like NGS, circularization of DNA, and assembly of multi-fragment constructs for synthetic biology. In contrast, focused acoustic energy-mediated ligation done using a crowding agent (PEG) for 20 minutes at 20 degrees C. has a significantly higher yield of the 2-linker fragment product, e.g., about 40%, dramatically improving upon all the standard methods.

FIG. 6 shows results of experiments conducted to test different times and amounts of focused acoustic energy exposure during ligation. In addition to a reaction during which no focused acoustic energy was applied, focused acoustic energy was applied to ligation reactions a) approximately continuously over 5 minutes, and only during burst times of b) 1 sec, c) 5 sec and d) 10 sec within a 1 minute interval. Focused acoustic energy applied during 1 sec, 5 sec, and 15 sec burst times per minute were applied in 15, 10 and 5 total treatments, respectively, e.g., the 1 second burst treatment occurred over a total of 15 minutes with 1 second bursts of acoustic energy applied during each minute of the 15 minute total treatment period. The reaction was done in a Covaris oneTUBE-10 and focused acoustic energy was provided by the E220 (Covaris) set at 20 PIP, 50% duty factor, and 100 cycles per burst. When short, periodical bursts of focused acoustic energy are applied to the ligation reaction, the 2-linker yield is highest with 1 second bursts compared to constant acoustic energy, longer acoustic energy bursts, or no acoustic energy. It is believed that shorter bursts create a turbulent, mixing environment which increases fragment end collision rate but also allows for binding in non-turbulent conditions. Thus, when the bursts are too long, or constant, it is believed that yield is lowered because of either solution heating or high molecular movement limiting enzyme binding.

### Example 3: Focused Acoustic Energy-Mediated Ligation is Improved by Adding a Crowding Agent

Experiments were conducted to assess the effect of a crowding agent on focused acoustic energy-mediated ligation, and it was found that adding a crowding agent, such as glycerol and PEG, increases the 2-linker product yield. As shown in FIG. 7, increasing the wt % of the crowding agent in the buffer had a moderate improvement on 2-linker yield, but the size of the crowding agent led to a significant improvement of 2-linker yield, particularly with the 6 k PEG. The size of the crowding agent is thought to influence DNA diffusion throughout the solution, so the optimal crowding agent may change with fragment size. The amount, or wt %, of the crowding agent is thought to increase the local concentration of enzyme, linker, and fragment, but can limit diffusion and change the rigidity of the DNA in solution.

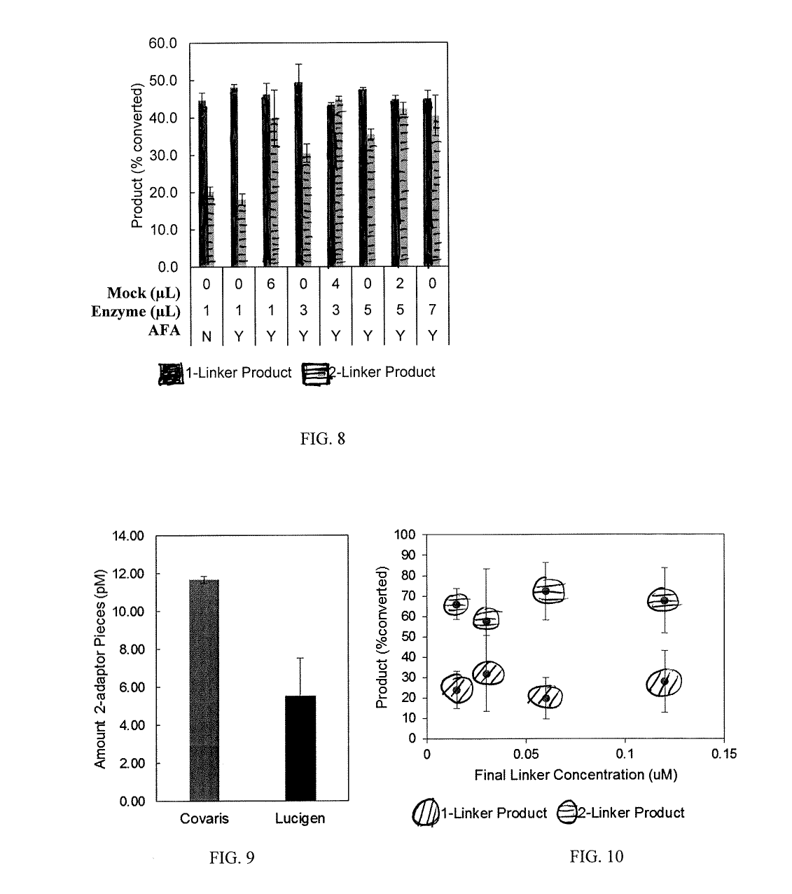

To further support that crowding agents combined with focused acoustic energy improve ligation, experiments were performed in which enzyme amount in the reaction was increased along with increasing the amount of crowding agent (e.g., glycerol). T4 ligase was supplied in a 50% glycerol solution, so a mock enzyme solution was made of 50 w/v % glycerol in water (Sigma). In FIG. 8, it is shown that adding a mock enzyme, essentially increasing the solution viscosity, is just as effective at increasing 2-linker fragments as increasing the total amount of enzyme in the solution. Thus, in turbulent mixing, such as that observed with focused acoustic energy application, local depletion of reactant is less of a problem and the DNA should have more interactions.

### Example 4: Focused Acoustic Energy-Mediated Ligation can be Used to Improve Library Preparation for NGS

Focused acoustic energy was integrated into a library preparation process using the NxSeq AmpFree Low DNA Library Kit (Lucigen) to demonstrate that focused acoustic energy-mediated ligation could be applied towards improving library preparation for NGS. 200 ng of gDNA (Promega) was sheared at 20° C. to an average size of 400 bp in 10 μL of TE buffer in a Covaris oneTUBE-10 on an LE220 machine (Covaris) set to 200 PIP, 25% duty factor, 50 cycles per bursts, and 1 mm dither at 20 m/s in the y-direction for 30 seconds. (In this regard, it should be noted that suitable focused acoustic energy may be used to shear DNA or other nucleic acids that are later subjected to a ligation reaction that also employs suitable focused acoustic energy.) A-tailing and end repair on the sheared gDNA was done on the Nexus GSX1 thermocycler set to 25° C. for 20 minutes and 72° C. for 20 minutes using the Lucigen enzyme mix. These products were mixed with Lucigen adaptors and ligase in accordance with the manufacturer's protocol. For the experiment results marked “Covaris,” ligation was performed in a oneTUBE-10 on the E220 (Covaris) set at 20° C. and programmed to give 1 second bursts of 20 PIP, 50% duty factor, and 100 cycles per burst every minute for 30 minutes. For the results marked “Lucigen,” no focused acoustic energy was employed. Clean-up and size selection was done with AMPure XP beads (Beckman and Coulter, Brea, Ca), in accordance with the manufacturer's protocol. To quantify the yield of 2-adapter pieces of gDNA, the KAPA Library Quantification Kit was used (KAPA Biosystems, Wilmington, Mass.). DNA was diluted to 1:1000 and 1:10000 in 10 mM Tris-HCL at pH 8.5 and room temperature. A 10 μL reaction was setup using 6 μL of KAPA SYBR FAST qPCR Master Mix (2×) plus Primer Premix (10×) and 0.2 μL of 50×ROX Low and 4 uL of either the diluted DNA sample, the provided DNA standards, or non-template controls. These reactions were analyzed on the Applied Biosystems 7500 Fast instrument set to have an initial denaturation at 95° C. for 5 minutes, with 35 subsequent cycles of denaturation at 95° C. for 30 seconds and annealing/extension/acquisition at 60° C. for 45 seconds. Data analysis was done by interpolating the Cq points for the unknowns between those for the known standards.

As shown in FIG. 9, when focused acoustic energy is applied during the reaction (indicated at “Covaris” in FIG. 9), the product yield of 2-linker fragments is almost double the yield obtained using the suggested ligation parameters in the Lucigen protocol. Additionally, experiments were performed to determine whether high yield of the 2-linker product can be maintained while reducing the initial concentration of linkers in solution. FIG. 10 shows the results of these experiments where four different initial concentrations of linkers were employed with a same starting amount of DNA fragments. In FIG. 10, the lowest linker input is about 50× lower than what is currently used in library prep kits. The results in FIG. 10 show that 2-linker product yield remained approximately constant for the different initial linker concentrations, suggesting that the linker to fragment ratio can be decreased using focused acoustic energy while still maintaining 2-linker product yield. Ultimately decreasing this ratio will reduce the clean-up needed and produce higher quality reads for NGS. Both results suggest that focused acoustic energy-mediated ligation can be applied towards improving library preparation for NGS. Downstream this would lead to reduced PCR amplification.

### REFERENCES

Brownstein, M., Carpten, J., & Smith, J. (1996). Modulation of non-templated nucleotide addition by Taq DNA polymerase: primer modifications that facilitate genotyping. BioTechniques 1, 20(6), 1004-1006, 1008-1010.
Clark, J. (1988). Novel non-templated nucleotide addition reactions catalyzed by procaryotic and eucaryotic DNA polymerases. Nucleic Acids Res 1, 16(20), 9677-9686.
Gibson, D., Benders, G., Andrews-Pfannkoch, C., Denisova, E., Baden-Tillson, H., Zaveri, J., . . . Smith, H. (2008). Complete Chemical Synthesis, Assembly, and clinign of a Mycoplasma genitalium Genome. Science, 319, 1215-1220.
Gibson, D., Young, L., Chuang, R.-Y., Venter, J., Hutchison III, C., & Smith, H. (2009). Enzymatic assembly of DNA molecules up to several hundred kilobases. Nature Methods, 6(5), 343-345.
Hayashi, K., Nakazawa, M., Ishizaki, Y., Hiraoka, N., & Obayashi, A. (1986). Regulation of inter- and intramoleculkar ligation with T4 DNA ligase in the presence of polyethylene glycol. Nueic Acids Research, 14(19), 7617-7631. https://doi.org/10.1093/nar/gkv1356
Head, S., Komori, H., LaMere, S., Whisenant, T., Nieuwerburgh, F., Salomon, D., & Ordoukhanian, P. (2014). Library construction for next-generation sequencing: Overviews and challenges. bioTechniques, 56(2), 61-77.
Head, S., Komori, H., LaMere, S., Whisenant, T., Van Nieuwerburgh, F., Salomon, D., & Ordoukhanian, P. (2014). Library construction for next-generation sequencing: overviews and challenges. Biotechniques, 56(2), 61-64, 66, 68.
Hou, S., Ziebacz, N., Kalwarczyk, T., Kaminski, T., Wieczorek, S., & Holyst, R. (2011). Influence of nano-viscosity and depletion interactions on cleavage of DNA by enzymes in glycerol and poly(ethylene glycol) solutions: qualitative analysis. Soft Matter, 7, 3092-3099.
Hutchison III, C., Chuang, R.-Y., Noskov, V., Assad-Garcia, N., Deerinck, T., Ellisman, M., . . . Venter, J. (2016). Design and synthesis of a minimal bacterial genome. Science, 351(6280). Illumina. (2017). Effects of Index Misassignment on Multiplexing and Downstream Analysis.
Kershaw, C. J., & Keefe, R. T. O. (2012). Splint ligation of RNA with T4 DNA ligase, 941(2), 257-269. https://doi.org/10.1007/978-1-62703-113-4
Lehnman, I. (1974). DNA Ligase: Structure, Mechanism, and Function. Science, 186(4166), 790-797.
Pheiffer, B., & Zimmerman, S. (1983). Polymer-stimulated ligation:enhanced blunt- or cohesive-end ligation of DNA or deoxyribooligonucleotides by T4 DNA ligase in polymer solutions. Nucleic Acids Research, 11(22). https://doi.org/10.1093/nar/gkn907
Rossi, R., Montecucco, A., Ciarrocchi, G., & Biamonti, G. (1997). Functional characterization of the T4 DNA ligase: a new insight into the mechanism of action. Nucleic Acids Research 1, 25(11), 2106-2113.
Shore, D., Langowski, J., & Baldwin, R. L. (1981). DNA flexibility studied by covalent closure of short fragments into circles. Proceedings of the National Academy of Sciences of the United States of America, 78(8), 4833-7. https://doi.org/10.1073/pnas.78.8.4833
Shuman, S. (2009). DNA ligases: Progress and prospects. Journal of Biological Chemistry, 284(26), 17365-17369. https://doi.org/10.1074/jbc.R900017200
Shuman, S. (2009). DNA Ligases: Progress and Prospects. The Journal of Biological Chemistry, 284(26), 17365-17369.
Sinha, R., Stanley, G., Gulati, G., Ezran, C., Travaglini, K., Wei, E., . . . Weissman, I. (n.d.). Index Switching Causes “Spreading-Of-Signal” Among Multiplexed Samples In Illumina HiSeq 4000 DNA Sequencing. bioRxiv.
Tsai, S. Q., Nguyen, N. T., Malagon-Lopez, J., Topkar, V. V, Aryee, M. J., & Joung, J. K. (2017). CIRCLE-seq: a highly sensitive in vitro screen for genome-wide CRISPR-Cas9 nuclease off-targets. Nature Methods, 14(6), 607-614. https://doi.org/10.1038/nmeth.4278

1. A method for processing nucleic acid material, comprising:
providing a nucleic acid material in a vessel, the nucleic acid material including a plurality of nucleic acid fragments;
providing a plurality of adapter fragments in the vessel, each of the adapter fragments arranged to ligate with an end region of a corresponding nucleic acid fragment;
providing at least one enzyme in the vessel, the at least one enzyme arranged to aid in ligation of adapter fragments and/or nucleic acid fragments to corresponding nucleic acid fragments; and
exposing the vessel holding the plurality of nucleic acid fragments, the plurality of adapter fragments and the enzyme to focused acoustic energy adapted to aid in the enzyme-aided ligation of adapter fragments to the nucleic acid fragments.

2. The method of claim 1, further comprising providing a crowding agent in the vessel to increase a viscosity of a mixture of the nucleic acid material, adapter fragments and at least one enzyme.

3. The method of claim 2, wherein the crowding agent includes polyethylene glycol (PEG).

4. The method of claim 1, wherein the step of exposing the vessel includes exposing a mixture of the nucleic acid material, adapter fragments and at least one enzyme to focused acoustic energy for a period of no more than 10 seconds per minute, or no more than 5 seconds per minute.

5. The method of claim 4, wherein the focused acoustic energy has a peak incident power (PIP) of 20 W or less, a 50% duty factor, and a cycles per burst of 100.

6. The method of claim 1, wherein the step of exposing includes ligating adapters to opposite ends of DNA fragments to form double-ligated DNA fragments.

7. The method of claim 6, wherein the step of exposing further includes ligating ends of adapter fragments that are each ligated to a corresponding end of a nucleic acid fragment to form a circularized DNA segment.

8. The method of claim 1, wherein the step of exposing includes ligating first and second ends of a nucleic acid fragment to form a circularized DNA segment.

9. The method of claim 1, wherein the step of exposing results in at least 30% of the nucleic acid fragments having ligated adapters at opposite ends to form double-ligated DNA fragments.

10. The method of claim 9, wherein a total ligation reaction time during which adapter fragments are permitted to ligate to nucleic acid fragments is less than 1 hour.

11. The method of claim 9, wherein the step of exposing results in at least 40% of the nucleic acid fragments having ligated adapters at opposite ends to form double-ligated DNA fragments.

12. The method of claim 11, wherein a total ligation reaction time during which adapter fragments are permitted to ligate to nucleic acid fragments is less than 1 hour.

13. The method of claim 1, wherein a total volume of the nucleic acid fragments, adapter fragments, and at least one enzyme is less than 100 microliters.

14. The method of claim 1, wherein a molar ratio of adapter fragments to nucleic acid fragments is less than 50:1.

15. The method of claim 9, wherein a molar ratio of adapter fragments to nucleic acid fragments is less than 50:1.

16. The method of claim 1, wherein a molar ratio of adapter fragments to nucleic acid fragments is less than 10:1.

17. The method of claim 9, wherein a molar ratio of adapter fragments to nucleic acid fragments is less than 10:1.

18. The method of claim 1, wherein a molar ratio of adapter fragments to nucleic acid fragments is 1:1 to 5:1.

19. The method of claim 9, wherein a molar ratio of adapter fragments to nucleic acid fragments is 1:1 to 5:1.

20. A method for processing nucleic acid material, comprising:
providing a nucleic acid material in a vessel, the nucleic acid material including a plurality of nucleic acid fragments with each nucleic acid fragment having first and second ends;
providing at least one enzyme in the vessel, the at least one enzyme arranged to aid in ligation of ends of nucleic acid fragments to corresponding ends of nucleic acid fragments; and
exposing the vessel holding the plurality of nucleic acid fragments and the at least one enzyme to focused acoustic energy adapted to aid in the enzyme-aided ligation of ends of the nucleic acid fragments to each other.

21. The method of claim 20, wherein the step of exposing includes ligating first and second ends of a nucleic acid fragment to form a circularized DNA segment.

22. The method of claim 20, further comprising providing a plurality of adapter fragments in the vessel, each of the adapter fragments arranged to ligate with an end of a corresponding nucleic acid fragment; and
wherein the step of exposing includes ligating ends of adapter fragments to corresponding ends of a nucleic acid fragment.

23. The method of claim 22, wherein the step of exposing further includes ligating free ends of adapter fragments that each have a ligated end ligated to a corresponding end of a nucleic acid fragment to form a circularized DNA segment.

24. The method of claim 20, further comprising providing a crowding agent in the vessel to increase a viscosity of a mixture of the nucleic acid material and at least one enzyme.

25. The method of claim 24, wherein the crowding agent includes polyethylene glycol (PEG).

26. The method of claim 20, wherein the step of exposing the vessel includes exposing a mixture of the nucleic acid material and at least one enzyme to focused acoustic energy for a period of no more than 10 seconds per minute.

27. The method of claim 26, wherein the focused acoustic energy has a peak incident power (PIP) of 20 W or less, a 50% duty factor, and a cycles per burst of 100.

28. The method of claim 26, wherein the step of exposing the vessel includes exposing a mixture of the nucleic acid material and at least one enzyme to focused acoustic energy for a period of no more than 5 seconds per minute.

29. The method of claim 20, wherein the step of exposing includes ligating adapters to the first and second ends of nucleic acid fragments to form double-ligated nucleic acid fragments.

30. The method of claim 29, wherein the step of exposing results in at least 30% of the nucleic acid fragments having ligated adapters at opposite ends to form double-ligated nucleic acid fragments.

31. The method of claim 30, wherein a total ligation reaction time during which adapters are ligated to ends of the nucleic acid fragments is less than 1 hour.

32. The method of claim 20, wherein a total volume of the nucleic acid fragments and at least one enzyme is less than 100 microliters.

33. The method of claim 20, wherein the first or second ends of the nucleic acid fragments include one of blunt ends, sticky ends, and a single nucleotide overhang.

34. The method of claim 33, wherein the plurality of nucleic acid fragments includes a plurality of adapter fragments in the vessel, each of the adapter fragments arranged to ligate with an end of a corresponding nucleic acid fragment; and
wherein the step of exposing includes ligating ends of adapter fragments to corresponding ends of a nucleic acid fragment.

35. The method of claim 20, wherein the step of providing nucleic acid material includes shearing nucleic acid material using focused acoustic energy to form the plurality of nucleic acid fragments.
